(19) United States
(12) Patent Application Publication
(10) Pub. No.: US 2011/0270399 A1
(43) Pub. Date: Nov. 3, 2011

(54) Mechanical Apparatus and Method for Artificial Disc Fusion and Nucleus Replacement

(76) Inventors: Matthew Yurek, San Diego, CA (US); Hansen Yuan, Naples, FL (US); Anthony Yeung, Phoenix, AZ (US); Craig Litherland, Menlo Park, CA (US); Kurt Veeder, Danville, CA (US); Robert Huffman, San Diego, CA (US)

(21) Appl. No.: 12/916,061

(22) Filed: Oct. 29, 2010

Related U.S. Application Data
(63) Continuation-in-part of application No. 11/153,776, filed on Jun. 15, 2005, now Pat. No. 8,021,426.
Application No. 11/153,776 is a continuation-in-part of application No. 11/173,034, filed on Jul. 1, 2005, now Pat. No. 7,442,210.
Application No. 11/173,034 is a continuation-in-part of application No. 11/273,299, filed on Nov. 14, 2005, now Pat. No. 7,601,172.
Application No. 11/273,299 is a continuation-in-part of application No. 11/359,335, filed on Feb. 22, 2006, now Pat. No. 7,547,319.
Application No. 11/359,335 is a continuation-in-part of application No. 11/700,509, filed on Jan. 31, 2007.
Application No. 11/700,509 is a continuation-in-part of application No. 12/316,789, filed on Dec. 16, 2008, now Pat. No. 7,988,735.

(51) Int. Cl. A61F 2/44

(52) U.S. Cl. 623/17.16

(57) ABSTRACT

The present invention relates to a device and method to perform 1) disc fusion, 2) artificial replacement of the nucleus, 3) artificial replacement of the annulus, or 4) artificial replacement of both the nucleus and annulus. The device is designed to be placed into the intervertebral space following discectomy. The invention includes a delivery catheter and an substantially fixed sized mesh loop with a lumen within the mesh loop and a center hole. The mesh loop partially self expands diametrically in diameter upon removal of the introducer sheath and can be further expanded by mechanical means.

### CROSS-REFERENCES

The present application is a continuation-in-part of patent application Ser. No. 11/153,776 filed on Jun. 15, 2005, 11/173,034 filed on Jul. 1, 2005, 11/273,299 filed on Nov. 14, 2005, 11/359,335 filed on Feb. 22, 2006, the 11/700,509 filed on Jan. 31, 2007 and the 12/316,789 filed on Dec. 16, 2008. These applications are incorporated herein by this reference.

### FIELD OF THE INVENTION

The present invention generally relates to devices and methods for the repair of intervertebral discs. More, specifically, the present invention relates to devices and methods for the treatment of spinal disorders associated with the nucleus, annulus and intervertebral disc.

### BACKGROUND OF THE INVENTION

Intervertebral disc disease is a major worldwide health problem. In the United States alone almost 700,000 spine procedures are performed each year and the total cost of treatment of back pain exceeds $30 billion. Age related changes in the disc include diminished water content in the nucleus and increased collagen content by the 4^{th} decade of life. Loss of water binding by the nucleus results in more compressive loading of the annulus. This renders the annulus more susceptible to delamination and damage. Damage to the annulus, in turn, accelerates disc degeneration and degeneration of surrounding tissues such as the facet joints.

The two most common spinal surgical procedures performed are discectomy and spinal fusion. These procedures only address the symptom of lower back pain. Both procedures actually worsen the overall condition of the affected disc and the adjacent discs. A better solution would be implantation of an artificial disc for treatment of the lower back pain and to restore the normal anatomy and function of the diseased disc.

The concept of a disc prosthesis dates back to a French patent by van Steenbrugghe in 1956. 17 years later, Urbaniak reported the first disc prosthesis implanted in animals. Since this time, numerous prior art devices for disc replacement have been proposed and tested. These are generally divided into devices for artificial total disc replacement or artificial nucleus replacement. The devices proposed for artificial total disc replacement, such as those developed by Kostuik, that generally involve some flexible central component attached to metallic endplates which may be affixed to the adjacent vertebrae. The flexible component may be in the form of a spring or alternatively a polyethylene core (Marnay). The most widely implanted total artificial disc to date is the Link SB Charite disc which is composed of a biconvex ultra high molecular weight polyethylene spacer interfaced with two endplates made of cobalt-chromium-molybdenum alloy. Over 2000 of these have been implanted with good results. However device failure has been reported along with dislocation and migration. The Charite disc also requires an extensive surgical dissection via an anterior approach.

The approach of artificial nucleus replacement has several obvious advantages over artificial total disc replacement. By replacing only the nucleus, it preserves the remaining disc structures such as the annulus and endplates and preserves their function. Because the annulus and endplates are left intact, the surgical procedure is much simpler and operative time is less. Several nuclear prostheses can be placed via a minimally invasive endoscopic approach. The nucleus implant in widest use today is the one developed by Raymedica (Bloomington, Minn.) which consists of a hydrogel core constrained in a woven polyethylene jacket. The pellet shaped hydrogel core is compressed and dehydrated to minimize size prior to placement. Upon implantation the hydrogel begins to absorb fluid and expand. The flexible but inelastic jacket permits the hydrogel to deform and reform in response to compressive forces yet constrain the horizontal and vertical expansion (see U.S. Pat. Nos. 4,904,260 and 4,772,287 to Ray). Other types of nuclear replacement have been described which include either an expansive hydrogel or polymer to provide for disc separation and relieve compressive load on the other disc components (see U.S. Pat. No. 5,192,326 to Boa). Major limitations of nuclear prostheses are that they can only be used in patients in whom disc degeneration is at an early stage because they require the presence of a competent natural annulus. In discs at later stages of degeneration the annulus is often torn, flattened and/or delaminated and may not be strong enough to provide the needed constraint. Additionally, placement of the artificial nucleus often requires access through the annulus. This leaves behind a defect in the annulus through which the artificial nucleus may eventually extrude compressing adjacent structures. What is clearly needed is a replacement or reinforcement for the natural annulus which may be used in conjunction with these various nuclear replacement devices.

Several annular repair or reinforcement devices have been previously described. These include the annulus reinforcing band described by U.S. Pat. No. 6,712,853 to Kuslich, which describes an expansile band pressurized with bone graft material or like, expanding the band. U.S. Pat. No. 6,883,520B2 to Lambrecht et al, describes a device and method for constraining a disc herniation utilizing an anchor and membrane to close the annular defect. U.S. patent application Ser. No. 10/676,868 to Slivka et al. describes a spinal disc defect repair method. U.S. Pat. No. 6,806,595 B2 to Keith et al. describes disc reinforcement by implantation of reinforcement members around the annulus of the disc. U.S. Pat. No. 6,592,625 B2 to Cauthen describes a collapsible patch put through an aperture in the sub-annular space. U.S. patent application Ser. No. 10/873,899 to Milbocker et al. describes injection of in situ polymerizing fluid for repair of a weakened annulus fibrosis or replacement or augmentation of the disc nucleus.

Each of these prior art references describes devices or methods utilized for repair of at least a portion of the diseased annulus, replacement of the damaged nucleus or conducting a spinal fusion. What is clearly needed is an improved spinal disc device and method capable of reinforcing the entire annulus circumferentially and/or replacing a damaged nucleus. In addition what is needed is an improved spinal disc device and method for performing spinal fusions. Additionally, what is clearly needed is a spinal disc device and method which may be easily placed into the intervertebral space and made to conform to this space. Furthermore, what is clearly needed is an improved spinal disc device and method capable of reinforcing the entire annulus that may be utilized either in conjunction with an artificial nucleus pulposis or may be used as a reinforcement for the annulus fibrosis and as an artificial nucleus pulposis.

### SUMMARY OF THE INVENTION

The present invention addresses this need by providing improved spinal disc device and methods for the treatment of intervertebral disc disease. The improved device and methods of the present invention specifically address disc related pain but may have other significant applications not specifically mentioned herein. For purposes of illustration only, and without limitation, the present invention is discussed in detail with reference to the treatment of damaged discs of the adult human spinal column.

As will become apparent from the following detailed description, the improved spinal disc device and methods of the present invention may reduce if not eliminate back pain while maintaining near normal anatomical motion. The present invention relates to devices and methods which may be used to reinforce or replace the native annulus, replace the native nucleus, replace both the annulus and nucleus or facilitate fusion of adjacent vertebrae. The devices of the present invention are particularly well suited for minimally invasive methods of implantation.

The spinal disc device is a catheter based or cannula based device with a unique delivery and expansion system which is placed into the intervertebral space following discectomy performed by either traditional surgical or endoscopic approaches. The distal end of the catheter is comprised of a fixed sized loop or mesh that is removably attached to a delivery tubular member using a locking collar assembly. Coaxially within the delivery tubular member is a delivery tubular member. The substantially flatten loop shaped mesh or toroidal shaped mesh is released from the jaws of the collar tubular member by retraction of collar tubular member over the delivery tubular member. The substantially flatten loop shaped mesh or toroidal shaped mesh may be formed of a woven, knitted, embroidered or braided material and may be made of PEEK (polyetheretherketone), Nylon, Dacron, synthetic polyamide, polypropylene, expanded polytetrafluoroethylene (e-PTFE), polyethylene and ultra-high molecular weight fibers of polyethylene (UHMWPE) commercially available as Spectra™ or Dyneema™, as well as other high tensile strength materials such as Vectran™, Kevlar™, natural or artificially produced silk and commercially available suture materials used in a variety of surgical procedures or a combination of these polymeric materials may be utilized. Alternatively the substantially flatten loop shaped mesh or toroidal shaped mesh portion of the catheter may be made of a biodegradable or bioabsorbable material such as resorbable collagen, LPLA (poly(l-lactide)), DLPLA (poly(dl-lactide)), LPLA-DLPLA, PGA (polyglycolide), PGA-LPLA or PGA-DLPLA, polylactic acid and polyglycolic acid which is broken down and bioabsorbed by the patient over a period of time. Alternatively the substantially flattened loop shaped mesh or toroidal shaped mesh may be formed from metallic materials, for example, stainless steel, elgiloy, Nitinol, or other biocompatible metals. Further, it is anticipated that the substantially flattened loop shaped mesh or toroidal shaped mesh b could be made from a flattened tubular knit, weave, mesh or foam structure. Again, a combination of these plastic, metal, or resorbable materials may be utilized in fabricating the present invention.

The substantially flattened loop shaped mesh or toroidal shaped mesh is formed such that one end of the loop feeds into its other end (invaginating), similar to a snake eating its own tail, forming the shape of a toroid or a substantially flatten loop mesh with an inner chamber and an inside hole section. The outer loop or mesh and the inner loop or mesh is sewn together using a thread design which yields a mesh or loop with a specific circumference size.

The present invention consists of a device and method, whereby the substantially flattened loop shaped mesh or toroidal shaped mesh is first delivered and fully expanded within the vertebral space to the limits of the inner portion of the native annulus to artificially replace all or a portion of a damaged nucleus.

The present invention consists of a device and method, whereby the substantially flattened loop shaped mesh or toroidal shaped mesh is first delivered and expanded within the vertebral space to the limits of the inner portion of the native annulus and then allograph materials are delivered into the center of the substantially flattened loop shaped mesh or toroidal shaped mesh.

The present invention consists of a device and method, whereby the substantially flattened loop shaped mesh or toroidal shaped mesh is first delivered and expanded within the vertebral space to the limits of the inner portion of the native annulus and then an injection of polymeric or hydrogel or like material is conducted to reinforce or artificially replace the native annulus.

The present invention also consists of a device and method, whereby the substantially flattened loop shaped mesh or toroidal shaped mesh is first delivered within the vertebral space and into the area of the nucleus, which may have been previously removed, and expanded to the limits of the outer portion of the area of the native nucleus and then injected with a polymer or hydrogel or like material conducted to reinforce or artificially replace the native nucleus.

The present invention also consists of a device and method, whereby the substantially flattened loop shaped mesh or toroidal shaped mesh is first delivered within the vertebral space and expanded within the vertebral space to the limits of the outer portion of the native annulus and then an injection of polymeric or hydrogel material is conducted to reinforce or artificially replace the native annulus.

Alternately, the present invention is delivered into the nucleus area and expanded to the limits of the outer portion of the native nucleus or an artificial nucleus concurrently placed and then an injection of polymeric or hydrogel material is conducted to reinforce or artificially replace or reinforce the nucleus.

The present invention also consists of a device and method, whereby the substantially flattened loop shaped mesh or toroidal shaped mesh is first delivered within the vertebral space and expanded within the vertebral space to the limits of the outer portion of the native annulus and then an injection of bone chips, autograft, allograft or osteoconductive/osteoinductive materials for spinal fusion is applied.

The present invention and variations of its embodiments is summarized herein. Additional details of the present invention and embodiments of the present invention may be found in the Detailed Description of the Preferred Embodiments and Claims below. These and other features, aspects and advantages of the present invention will become better understood with reference to the following descriptions and claims.

### BRIEF DESCRIPTION OF THE DRAWINGS

FIG. 1 is a partial sectional view the present invention showing the substantially flattened loop shaped mesh or toroidal shaped mesh engaged to the collar jaw means to a first and second tubular members and displaying the sewn inside and outside mesh.

FIG. 2 is a side view taken from FIG. 1 of the tubular member showing a sewing pattern which creates a fix-sized substantially tubular toroidal or flat loop shaped mesh.

FIG. 3 is a partial sectional view of the present invention showing the coaxial relationship of the fill/delivery tubular member within the collar tubular member with substantially flattened loop shaped mesh or toroidal shaped mesh engaged to the jaws of the collar tubular member.

FIG. 4 is a top-side view of a first embodiment of the collar tubular member.

FIG. 5 is a top-side view of a first embodiment of the fill/delivery tubular member.

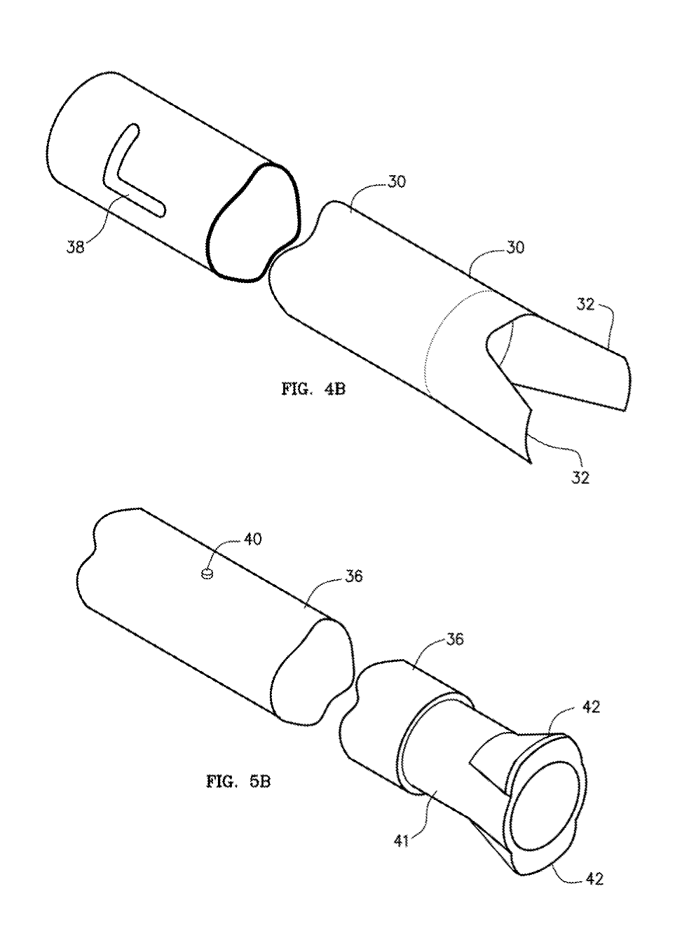

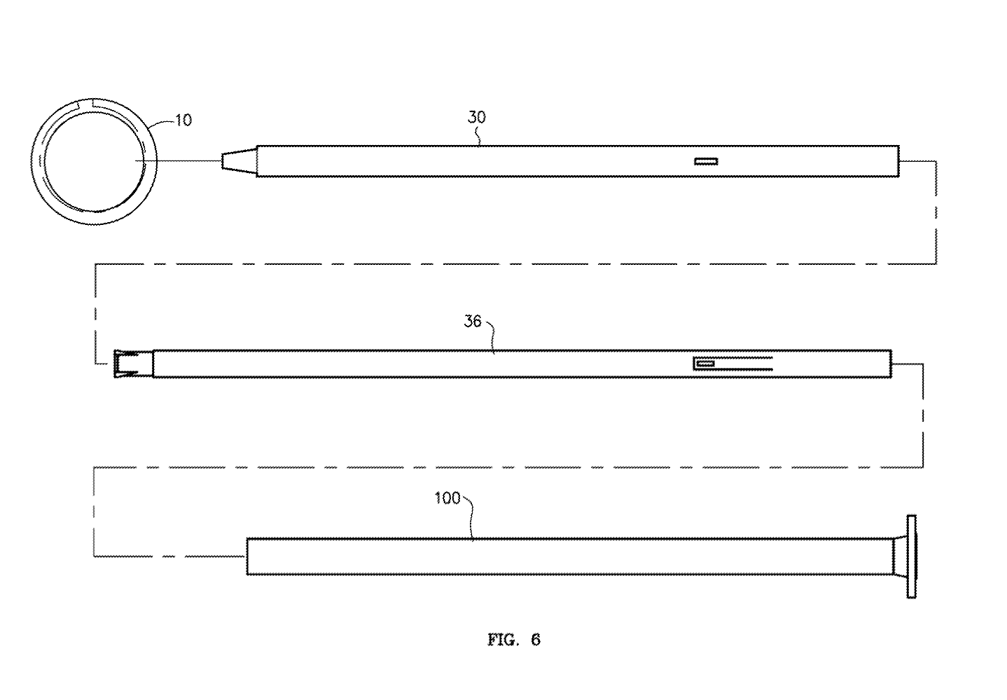

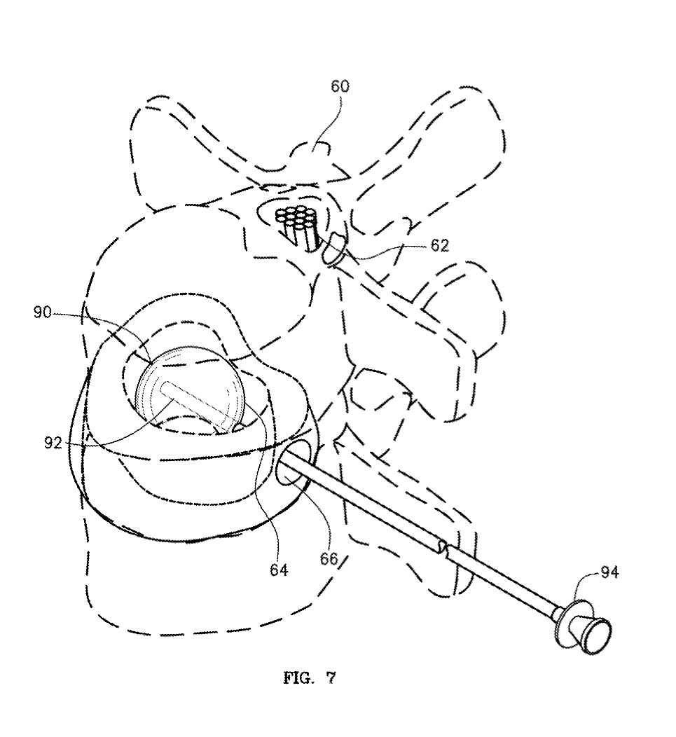

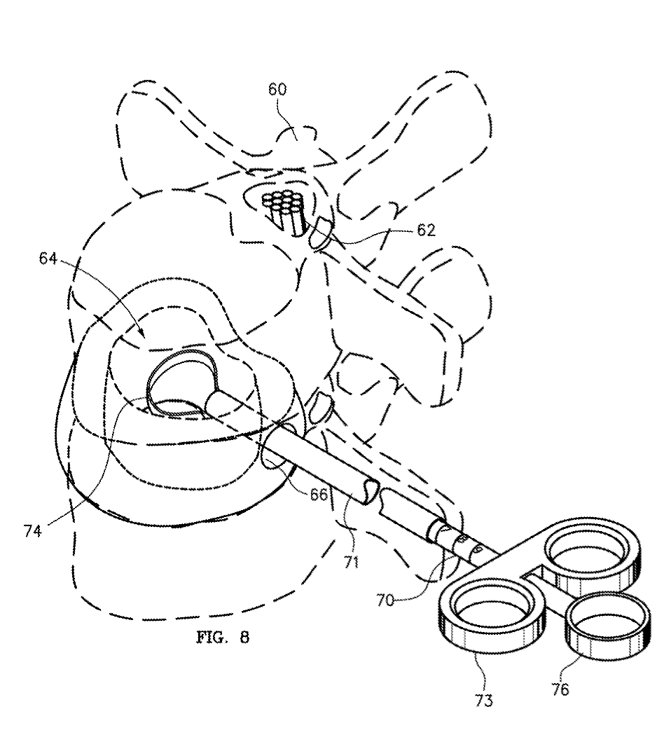

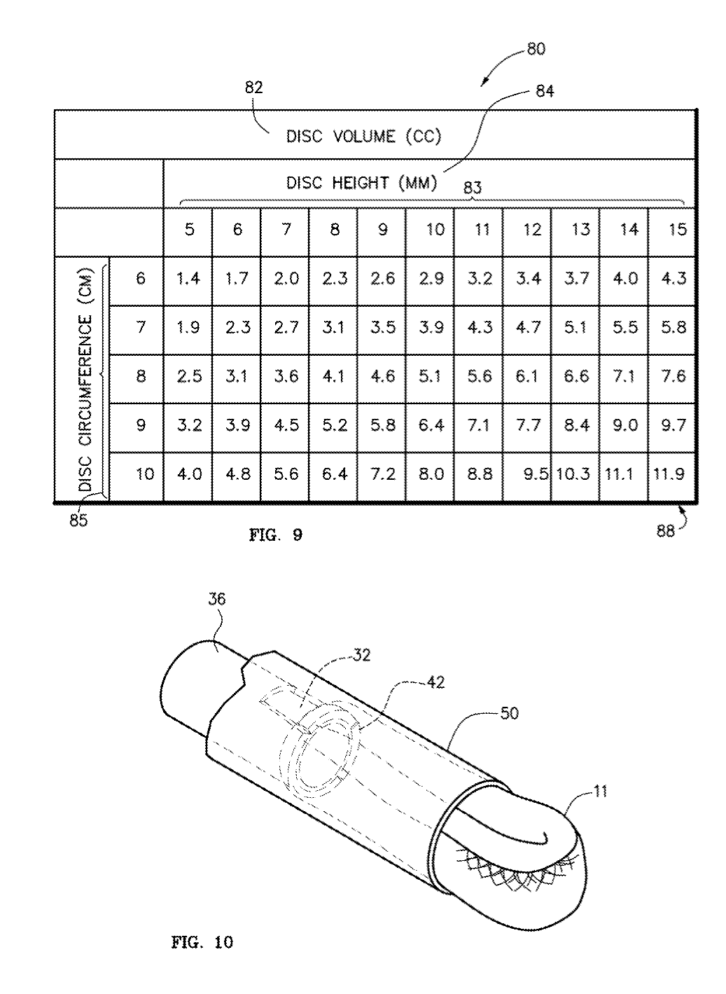

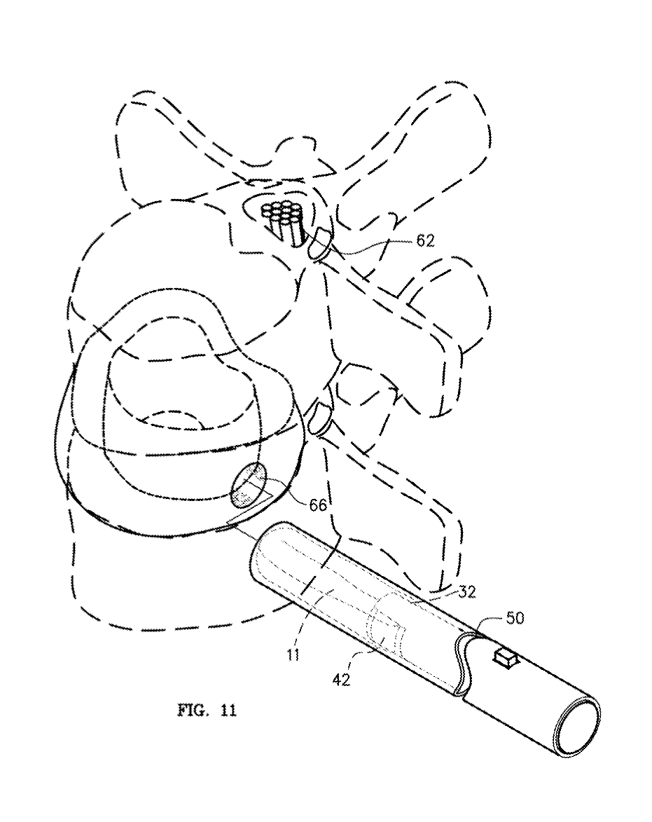

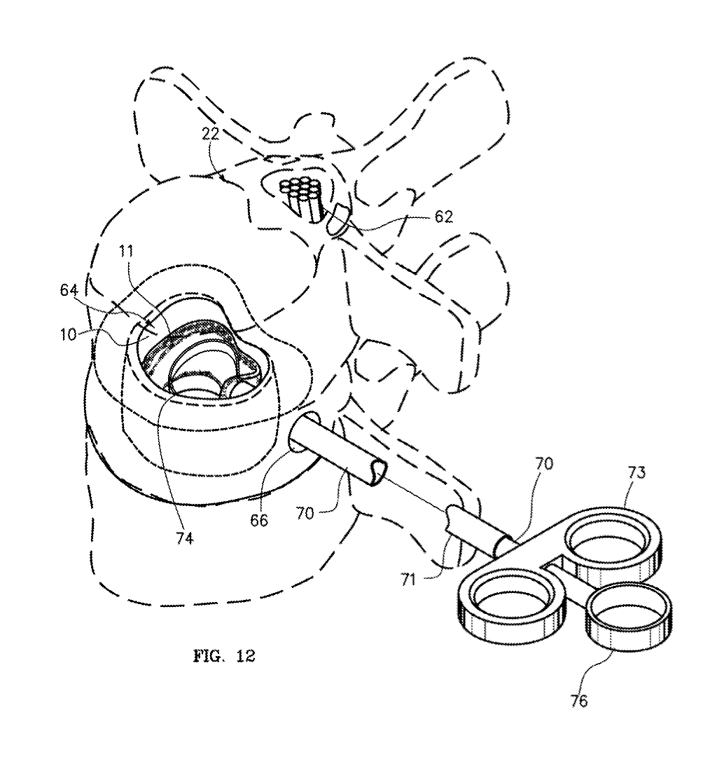

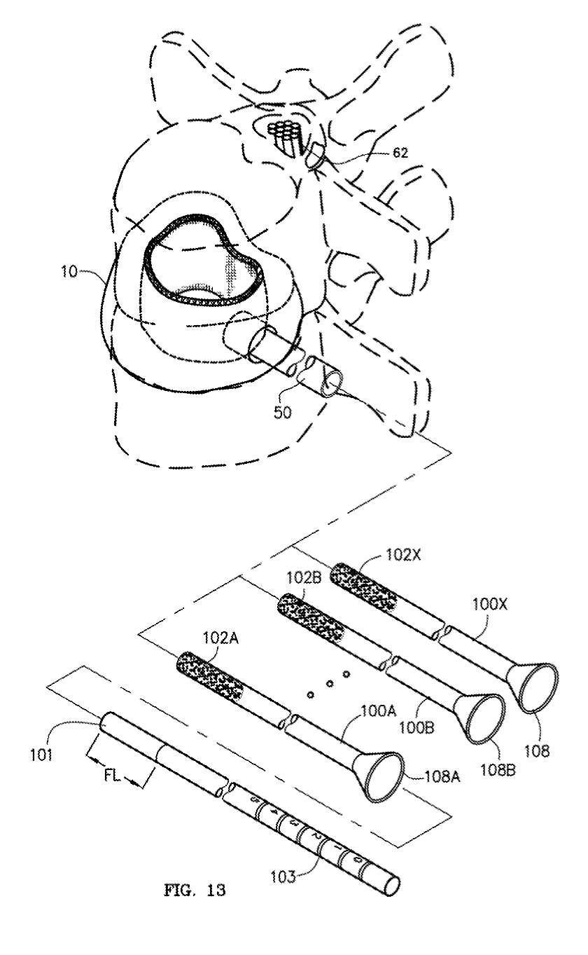

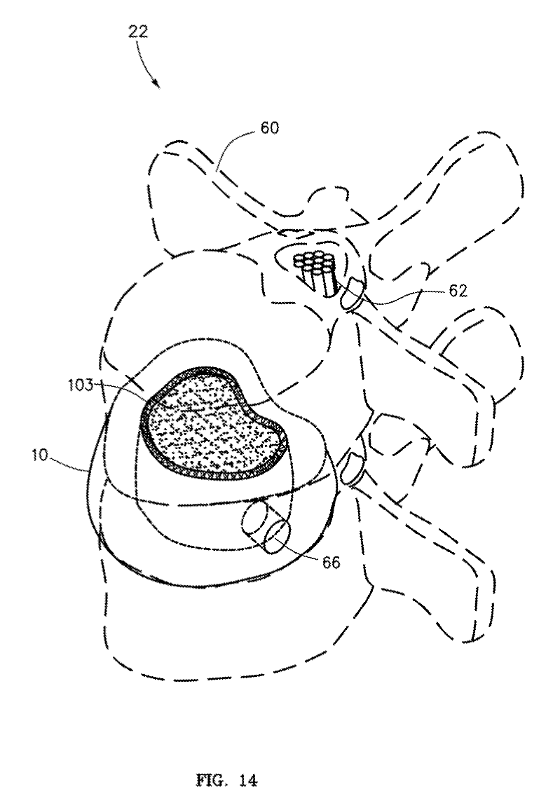

FIG. 4a is a top-side view of a second embodiment of the collar tubular member.

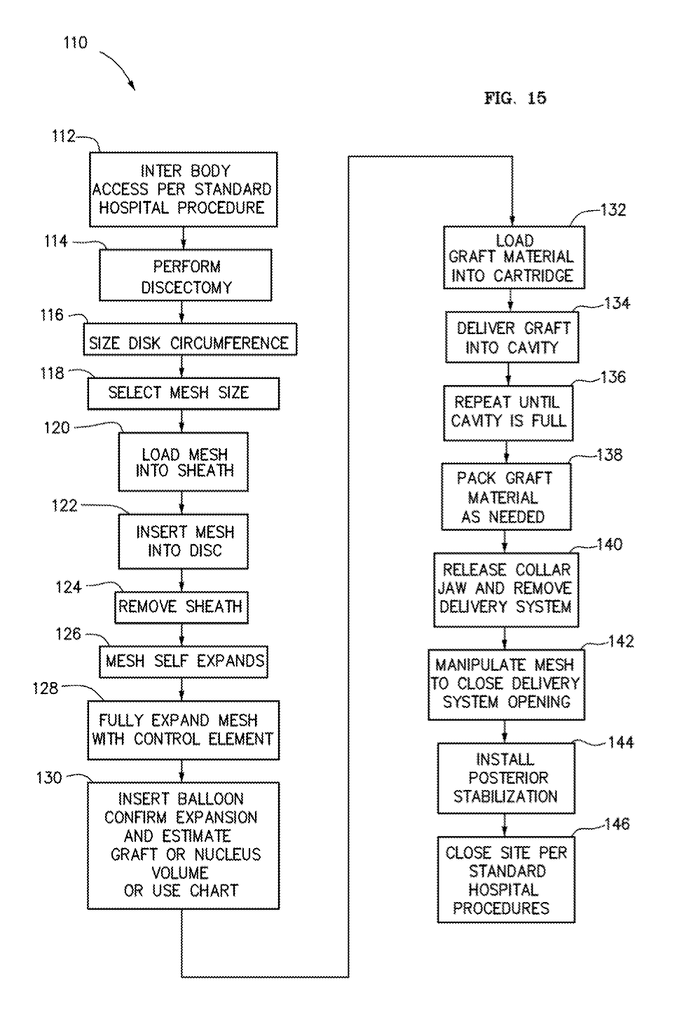

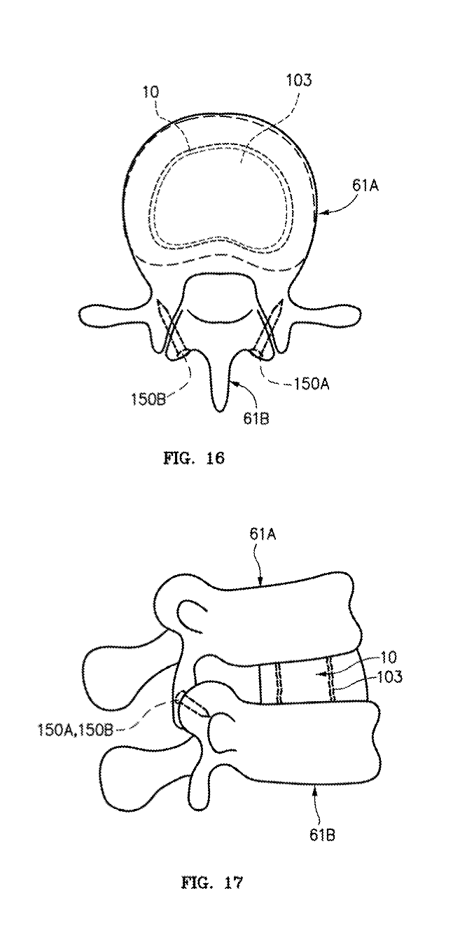

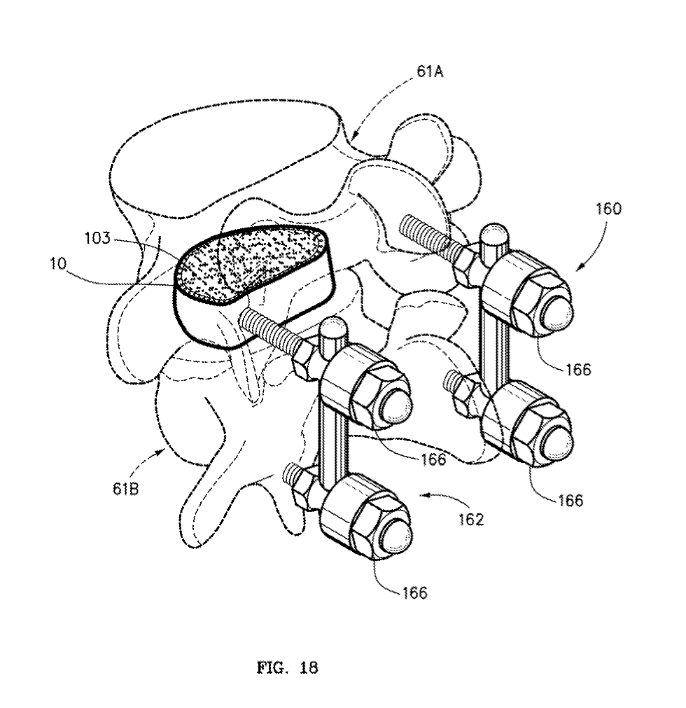

FIG. 5a is a top-side view of a second embodiment of the fill/delivery tubular member.

FIG. 4b is a top-side view of a third embodiment of the collar tubular member.

FIG. 5b is a top-side view of a third embodiment of the fill/delivery tubular member.

FIG. 6 is a side view of the collar tubular member, the fill/delivery tubular member, the substantially flattened loop shaped mesh or toroidal shaped mesh and a sheath used to constrain the substantially flattened loop shaped mesh or toroidal shaped mesh.

FIG. 7 is a partial cross-sectional view of an intervertebral disc with an optional balloon that confirms the extent and volume of the discectomy inserted through an access opening.

FIG. 8 is a partial cross-sectional view of an intervertebral disc with the removable control element inserted through an access opening.

FIG. 9 is a perspective view of a disc space volume chart for use with the present invention.

FIG. 10 is a partial cross-section view of the distal end the present invention showing the substantially flattened loop shaped mesh or toroidal shaped mesh in a folded configuration and inserted within the distal end of the delivery sheath member.

FIG. 11 is a partial cross-sectional of the treatment configuration of the present invention in position to be inserted into an access hole in an intervertebral disc.

FIG. 12 is a partial cross-section view of the present invention with substantially flattened loop shaped mesh or toroidal shaped mesh deployed with the disc space and the removable control element deployed to expand the substantially flattened loop shaped mesh or toroidal shaped mesh against the inner wall of the disc space.

FIG. 13 is a partial cross-section view of the present invention substantially flattened loop shaped mesh or toroidal shaped mesh fully expanded with the disc space and showing a series of autograft of allograft fill tube cartridges.

FIG. 14 is a partial cross-section of a treated intervertebral disc showing the substantially flattened loop shaped mesh or toroidal shaped mesh filled with autograft or allograft material with the disc space.

FIG. 15 is a flowchart depicting the general sequence of steps used with the present invention and its accessory components.

FIG. 16 is a partial sectional top view of an intervertebral disc showing the substantially flattened loop shaped mesh or toroidal shaped mesh filled with autograft or allograft material within the disc space and two successive discs affixed using a plurality of facet screws.

FIG. 17 is a partial sectional side view a first intervertebral disc and a second intervertebral discs affixed with a plurality of facet screws

FIG. 18 is posterior view of an intervertebral disc showing the substantially flattened loop shaped mesh or toroidal shaped mesh filled with autograft or allograft material within the disc space and two successive discs affixed using a pair of pedicle screws for posterior support.

### DESCRIPTION OF THE PREFERRED EMBODIMENTS

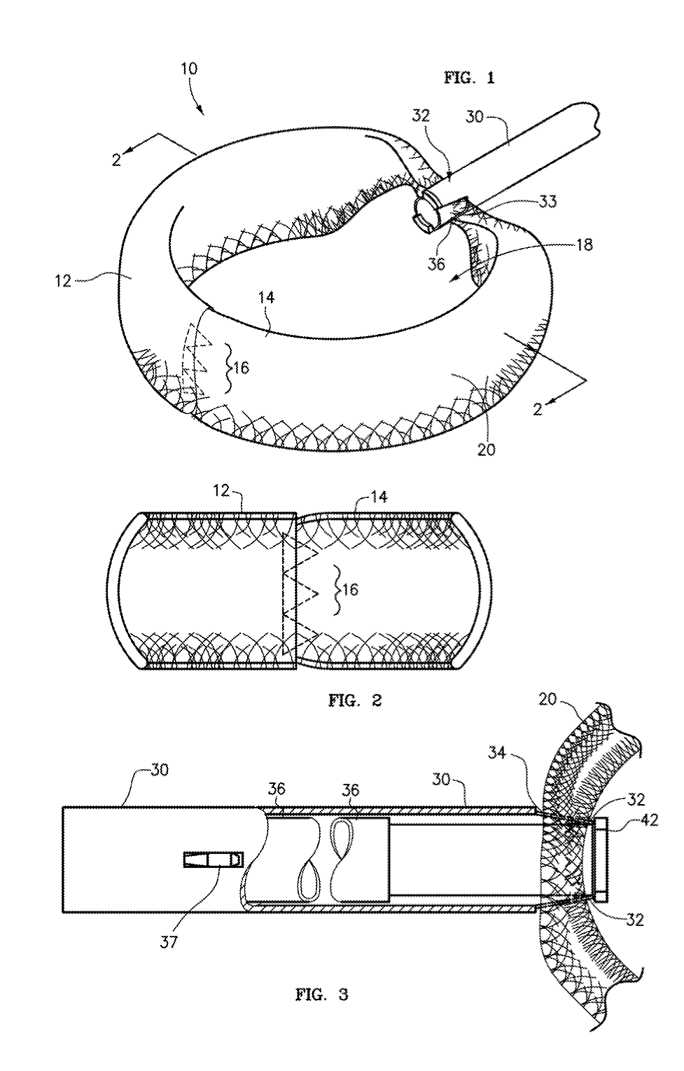

FIG. 1 shows a partial sectional view of the present invention showing the unfilled substantially flattened loop shaped mesh or toroidal shaped mesh 10 that is engaged to a collar jaw means to a first and second tubular members and displaying the sewn inside and outside mesh located on the distal end of the tubular members 30 and 36. The substantially flattened loop shaped mesh or toroidal shaped mesh 10 is braided, knitted, woven or embroidered and substantially flatten loop mesh or toroidal shape mesh (also sometimes referred herein as the fixed sized mesh loop) and is comprised of an inner mesh 14 and an outer mesh 12 whereby a portion of the inner mesh 14 is inserted into a portion of the outer mesh 12 and held together with sewn area 16. This design results in a substantially flattened loop shaped mesh or toroidal shaped mesh 10 having a substantially flatten loop mesh in a toroidal configuration with an inner chamber 20 and inside centrally located hole area 18. The substantially flattened loop shaped mesh or toroidal shaped mesh 10 is fabricated as a knit, weave, embroidered, or braid and can be constructed from non-degradable materials. Suitable non-degradable materials for the substantially flattened loop shaped mesh or toroidal shaped mesh 10, include, but are not limited to, PEEK (polyetheretherketone), Nylon, Dacron, synthetic polyamide, polypropylene, expanded polytetrafluoroethylene (e-PTFE), polyetheretherketone (PEEK), polyethylene and ultra-high molecular weight fibers of polyethylene (UHMWPE) commercially available as Spectra™ or Dyneema™, as well as other high tensile strength materials such as Vectran™, Kevlar™, natural or artificially produced silk and commercially available suture materials used in a variety of surgical procedures. The substantially flattened loop shaped mesh or toroidal shaped mesh 10 fabricated as a weave, knit, embroider or braid and can be constructed from biodegradable or bioabsorbable materials. Suitable biodegradable and bioabsorbable materials for the expandable mesh or loop 150 include, but are not limited to, resorbable collagen, LPLA (poly(l-lactide)), DLPLA (poly(dl-lactide)), LPLA-DLPLA, PGA (polyglycolide), PGA-LPLA or PGA-DLPLA, and biodegradable sutures made from polylactic acid and polyglycolic acid.

In addition, for some embodiments, suitable metallic materials for the substantially flattened loop shaped mesh or toroidal shaped mesh 10 may be used that include, but are not limited to, stainless steel, cobalt-chrome alloy, titanium, titanium alloy, or nickel-titanium shape memory alloys, among others. It is further contemplated that the metallic mesh can be interwoven with non-resorbable polymers such as nylon fibers, polypropylene fibers, carbon fibers and polyethylene fibers, among others, to form a metal-polymer composite weave. Further examples of suitable non-resorbable materials include DACRON and GORE-TEX. One feature of the substantially flattened loop shaped mesh or toroidal shaped mesh 10 is that it needs to have pore sizes or openings that are small enough to hold the filling material or nucleus from extruding out and large enough to maintain flexibility, expansion characteristics, and transport of biological materials for incorporation or fusion.

For spinal delivery, a portion 33 of the substantially flattened loop shaped mesh or toroidal shaped mesh 10 is engaged using a locking jaw mechanism 32. The locking jaws 32 is located on the distal end of a collar tubular member 30 and holds the engaged portion 33 of the substantially flattened loop shaped mesh or toroidal shaped mesh 10 between the locking jaws 32 and a fill/delivery tubular member 36. The fill/delivery tubular member 36 is in coaxial association with the collar tubular member such that the two tubular members can move with respect to each other. Suitable metallic materials for the collar tubular member 30 and fill/delivery tubular member 36 may be used that include, but are not limited to, stainless steel, cobalt-chrome alloy, titanium, titanium alloy, or nickel-titanium shape memory alloys, among others. In addition, suitable polymeric materials for the collar tubular member 30 and fill/delivery tubular member 36 may be used that include, but are not limited to, PEEK (polyetheretherketone), Nylon, Dacron, ABS, acrylics, polyamide, polypropylene, expanded polytetrafluoroethylene (e-PTFE), polyethylene, ultra-high molecular weight polyethylene (UHMWPE), Kraton (polyisoprene), PET (Polyethylene terephthalate) and acetal.

FIG. 2 is a side view taken from FIG. 1 of the tubular member shows a portion of the inner mesh 14 is inserted into a portion of the outer mesh 12 and held together with sewn area 16, which creates a fix-sized substantially tubular toroidal or substantially flattened loop shaped mesh 10. To create the sewn area 16, a PEEK monofilament threaded pattern is utilized. The PEEK monofilament thread has a diameter that ranges from 0.001″ to 0.015″ with a preferred range of 0.004″ to 0.008″. A multifilament thread could also be used and would increase the flexibility of the braid. The pattern shown is a single straight zigzag configuration that travels from approximately the top of the sewn area 16 to approximately the bottom of the sewn area 16. The Applicants assert that there are numerous thread patterns that can be used, such as, but not limited to, double straight zigzag, single and double hourglass, single and double crosshatch patterns, and other patterns without deviating from the functional aspect required for the present invention. The substantially flattened loop shaped mesh or toroidal shaped mesh 10 could also be formed from a tubular braid, knit, weave, embroidered, or a flat knit or weave that has been sewn or melted formed together.

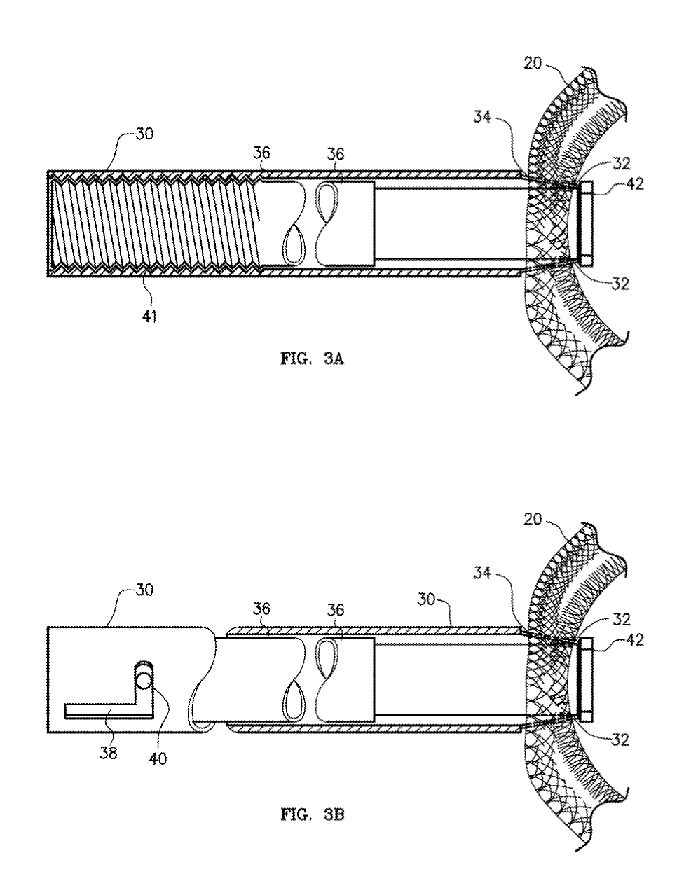

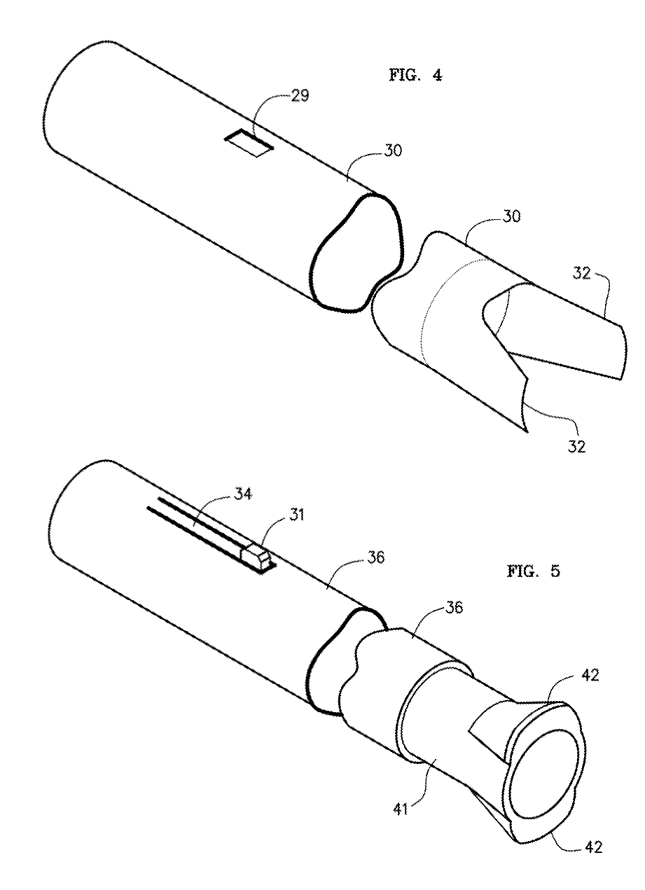

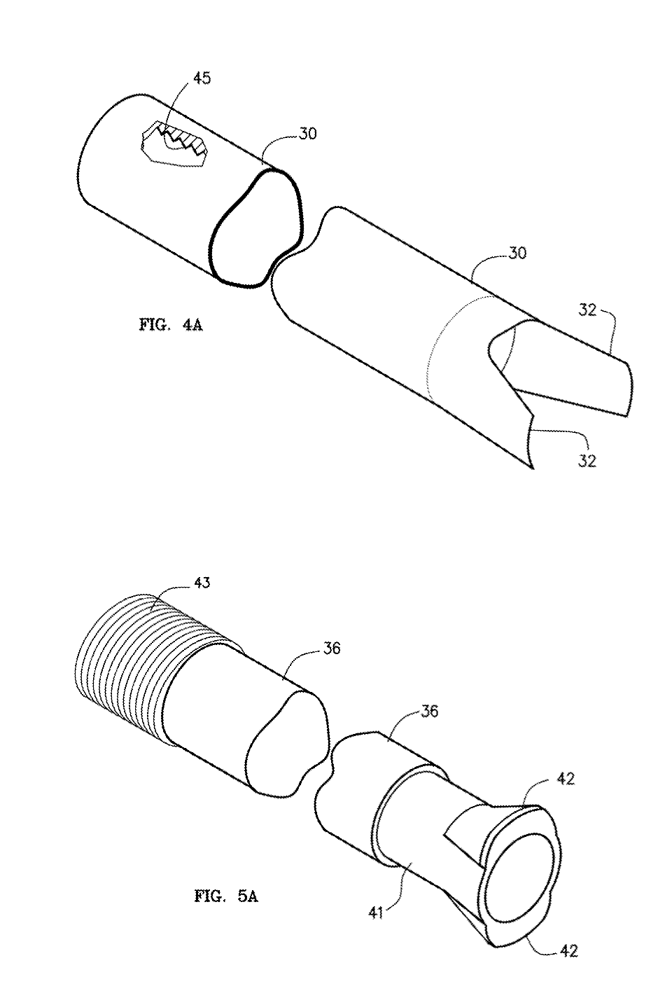

FIG. 3 is a partial sectional view of the present invention showing in more detail the slidable coaxial relationship of the fill/delivery tubular member 36 within the collar tubular member 30 with fixed size substantially flattened loop shaped mesh or toroidal shaped mesh 10 removably engaged to the locking jaws 32 of the collar tubular member 30. To aid in the removable engagement, a pair of raised ears 42 is located on the distal end of the fill/delivery tubular member 36. Also shown in this Figure is a locking mechanism 37 which, when properly employed, maintains the relative position of the fill/delivery tubular member 36 within the collar tubular member 30. As will be shown in FIGS. 4, 4a 4b, 5, 5a and 5b below there are several embodiments for achieving the locking functionality.

FIG. 4 is a top-side view of a first embodiment of the fill/delivery tubular member 36 showing a deflectable button 31 that is designed to enter and exit the cutout 29. The cutout 29 has a width that ranges from 0.075″ to 0.225″ with a preferred range of 0.125″ to 0.135″ and a length that ranges from 0.1″ to 0.5″ with a preferred range of 0.250″ to 0.300″. The deflectable button 31 is an integrated into the fill/delivery tubular member 36. The deflectable button 31 has a diameter that ranges from 0.070″ to 0.220″ with a preferred range of 0.120″ to 0.130″ and a height that ranges from 0.01″ to 0.1″ with a preferred range of 0.02″ to 0.050″ above the surface of the tubular members. The deflectable button 31 is attached or engaged to a deflectable elongated tab 34 in the fill/delivery tubular member 36. The deflectable tab 34 has a width that ranges from 0.001″ to 0.50″ with a preferred range of 0.01″ to 0.20″ and a length that ranges from 0.10″ to 3.0″ with a preferred range of 0.50″ to 1.5″. Both the cutout 29 and the deflectable elongated tab 34 are preferably fabricated by laser cutting but can be fabricated by other means, such as machining, wire electron discharge machining (EDM), stamping, etc. To aid in the removable engagement, a pair of raised ears 42 is located on the distal end of the fill/delivery tubular member 36. When the button 31 is in its relaxed extended state, the button 31 protrudes through the cutout 29 in the collar tubular member 30 positioned coaxially over the fill/delivery tubular member 36, restricting any rotational or longitudinal movement between the collar tubular member 30 and the fill/delivery tubular member 36. In this non-movement state, the collar tubular member 30 and the fill/delivery tubular member 36 are designed such that the jaws on the distal end on the collar tubular member 30 come in close contact with the outside surface of the distal end of the fill/delivery tubular member 36. In this non-movement state, the distal end of both tubular members, 30 and 36 are designed to engage a portion of the substantially flattened loop or toroidal shaped mesh 10. When the button is depressed, the button exits the cutout 29 and longitudinal movement between the collar tubular member 30 and the fill/delivery tubular member 36 is allowed and retraction of the collar tubular member proximally over the fill/delivery tubular member 36 releases the engaged portion of the substantially flattened loop or toroidal shaped mesh 10.

FIG. 4a is a top-side view of a second embodiment of the collar tubular member 36, male threads 43 that were cut into the fill/delivery tubular member 36 for screwably engaging the female threads 45 on inside surface of collar tubular member 30. Shown in more detail are the locking jaws 32.

FIG. 5a is a top-side view of a second embodiment of the fill/delivery tubular member 36. The male threads 43 are integrated into the outside surface of the fill/delivery tubular member 36 and the female threads are integrated within the inside surface of the collar tubular member 30. The female threads have a thread size that ranges from 4-40 to ½-16 with a preferred range of 10-32 to ⅜″-24. The male thread size matches the female thread size and could also be a metric size or custom thread sizes. The male threads 43 and the female threads are fabricated using standard screw technology. When the fill/tubular member 36 is rotated counter clockwise inside the collar tubular member 30, the distal end of the fill/deliver tubular member 36 moves proximally such that the distal ends of both the tubular members 30 and 36 are designed to engage a portion of the substantially flattened loop shaped mesh or toroidal shaped mesh 10. When the fill/delivery tubular member 36 is rotated clockwise inside the collar tubular member 30, the distal end of the fill/delivery tubular member 36 moves distally releasing the engaged portion of the substantially flattened loop shaped mesh or toroidal shaped mesh 10. It is anticipated by the Applicants that the rotation of the tubular members can be reversed, e.g. rotated counter-clockwise to release a portion and clockwise to engage a portion of the substantially flattened loop shaped mesh or toroidal shaped mesh 10.

FIG. 4b is a top-side view of a third embodiment of the collar tubular member 30 showing a “L” configured cutout 38 in the collar tubular member 30 for receiving a non-deflectable button 40. Shown in more detail are the locking jaws 32.

FIG. 5b is a top-side view of a third embodiment of the fill/delivery tubular member 36 showing a non-deflectable button 40 that is designed to track the “L” configured cutout 38. The “L” configured cutout 38 has a longitudinal width that ranges from 0.01″ to 0.075″ with a preferred range of 0.03″ to 0.05″ and a longitudinal length that ranges from 0.2″ to 0.5″ with a preferred range of 0.25″ to 0.275″. The “L” configured cutout 38 has a perpendicular width that ranges from 0.001″ to 0.075″ with a preferred range of 0.03″ to 0.05″ and a perpendicular length that ranges from 0.01″ to 0.25″ with a preferred range of 0.225″ to 0.265″. The non-deflectable button 40 is an integrated into the fill/delivery tubular member 36. The non-deflectable button 40 has a diameter that ranges from 0.010″ to 0.075″ with a preferred range of 0.03″ to 0.05″ and a height that ranges from 0.015″ to 0.035″ with a preferred range of 0.020″ to 0.040″. To aid in the removable engagement, a pair of raised ears 42 is located on the distal end of the fill/delivery tubular member 36. The “L” configured cutout 38 is fabricated by laser cutting, machining, wire electron discharge machining (EDM) or stamping out an “L” configuration. When the non-deflectable button 40 is in the perpendicular groove or track, any longitudinal movement between the collar tubular member 30 and the fill/delivery tubular member 36 is restricted. In this non-restricted state, the collar tubular member 30 and the fill/delivery tubular member 36 are designed such that the jaws on the distal end on the collar tubular member 30 come in close contact with the outside surface of the distal end of the fill/delivery tubular member 36. In this restricted state, the distal end of both tubular members, 30 and 36 are designed to engage a portion of the substantially flattened loop shaped mesh or toroidal shaped mesh 10. When the non-deflectable button 40 is moved to the longitudinal groove or track in the “L” configured cut out 38, longitudinal movement between the collar tubular member 30 and the fill/delivery tubular member 36 is allowed and retraction of the collar tubular member proximally over the fill/delivery tubular member 36 releases the engaged portion of the substantially flattened loop shaped mesh or toroidal shaped mesh 10.

FIG. 6 is a side view of the collar tubular member 30, the fill/delivery tubular member 36, the fixed-sized substantially flattened loop shaped mesh or toroidal shaped mesh 10 and a sheath 100. As shown by this Figure, the sheath 100 is coaxially inserted over the fill/delivery tubular member 36. The fill/delivery tubular member 36 coaxially is insertable within the collar tubular member 30. The collar tubular member 30 can be fabricated from suitable metallic materials that include, but are not limited to, stainless steel, cobalt-chrome alloy, titanium, titanium alloy, or nickel-titanium shape memory alloys, among others. In addition, suitable polymeric materials for the collar tubular member 30 may be used that include, but are not limited to, PEEK (polyetheretherketone), Nylon, Dacron, ABS, acrylics, polyamide, polypropylene, expanded polytetrafluoroethylene (e-PTFE), fluorinated ethylene propylene (FEP), polyethylene. ultra-high molecular weight polyethylene (UHMWPE), Kraton (polyisoprene), PET (Polyethylene terephthalate) and acetal. The fill/delivery tubular member 36 can be fabricated from suitable metallic materials that include, but are not limited to, stainless steel, cobalt-chrome alloy, titanium, titanium alloy, or nickel-titanium shape memory alloys, among others. In addition, suitable polymeric materials for the fill/delivery tubular member 36 may be used that include, but are not limited to, PEEK (polyetheretherketone), Nylon, Dacron, ABS, acrylics, polyamide, polypropylene, expanded polytetrafluoroethylene (e-PTFE), fluorinated ethylene propylene (FEP), polyethylene ultra-high molecular weight polyethylene (UHMWPE) Kraton (polyisoprene), PET (Polyethylene terephthalate) and acetal. The sheath 100 can be fabricated from suitable metallic materials that include, but are not limited to, stainless steel, cobalt-chrome alloy, titanium, titanium alloy, or nickel-titanium shape memory alloys, among others. In addition, suitable polymeric materials for the sheath may be used that include, but are not limited to, PEEK (polyetheretherketone), Nylon, Dacron, ABS, acrylics, polyamide, polypropylene, expanded polytetrafluoroethylene (e-PTFE), fluorinated ethylene propylene (FEP), polyethylene, ultra-high molecular polyethylene (UHMWPE), Kraton (polyisoprene), PET (Polyethylene terephthalate) and acetal. Also shown in this Figure is the fixed sized substantially flattened loop shaped mesh or toroidal shaped mesh 10. It is anticipated by the Applicants that the sheath 100 can be advanced over the fill/delivery tubular member 36, the collar tubular member 30 and retained substantially flattened loop shaped mesh or toroidal shaped mesh 10 thus constraining the substantially flattened loop shaped mesh or toroidal shaped mesh 10 into a reduced diameter delivery configuration as will be described in more detail in FIG. 10.

FIG. 7 is a partial cross-sectional view of an intervertebral disc 60 having spinal nerves 62 with an optional discectomy confirming contrast filled (volume measuring) balloon 90 mounted on a catheter shaft 92 with a proximally located inflation/deflation means 94. The balloon 90 and catheter shaft 92 are inserted through an access opening 66 into the dissected disc space.

The balloon 90 is flexible such that it can be deflated and contracted for insertion and removal through access opening 66 and then able to be inflated when within the disc space. Suitable materials for the balloon 90, include, but are not limited to, Nylon, Dacron, synthetic polyamide, polypropylene, fluorinated ethylene propylene (FEP), polyethylene, Pebax, silicone and urethane materials, Kraton (polyisoprene), PET (Polyethylene terephthalate) and acetal.

Suitable materials for the catheter shaft 92, include, but are not limited to, PEEK (polyetheretherketone), Nylon, Dacron, synthetic polyamide, polypropylene, polytetrafluoroethylene (PTFE), fluorinated ethylene propylene (FEP), polyetheretherketone (PEEK), polyethylene and ultra-high molecular weight fibers of polyethylene, Kraton (polyisoprene), PET (Polyethylene terephthalate) and acetal.

The balloon, when fully inflated within the disc space 64, can provide a fluoroscopic assessment of the dissection.

FIG. 8 is a partial cross-section of an intervertebral disc 60 and spinal nerves 62 with the removable control element 61 inserted through an access opening 66. The control element is comprised of an outer shaft 70 which is in coaxial relationship with an inner shaft 71 which is fitted with an expandable distal loop 74. The coaxial relationship between the outer shaft 70 and the inner shaft 71 is such that as the inner shaft 71 moves forward, the distal loop 74 expands and when the inner shaft is moved back, the distal loop 74 contracts. A thumb handle 76 is attached to the proximal end of the inner shaft 71 and a finger handle is attached to the proximal end of the outer shaft 70 for ergonomic assistance in moving the inner shaft forward and back.

The inner shaft 71 and outer shaft 70 can be fabricated from suitable metallic materials that include, but are not limited to, stainless steel, cobalt-chrome alloy, titanium, titanium alloy, or nickel-titanium shape memory alloys, among others. Suitable polymeric materials for inner shaft 71 and outer shaft 70, include, but are not limited to, polyetheretherketone (PEEK), Nylon, Dacron, synthetic polyamide, polypropylene, polyethylene, silicone and urethane materials. Suitable non-degradable materials for the distal loop 74, include, but are not limited to, PEEK (polyetheretherketone), Nylon, Dacron, synthetic polyamide, polypropylene, polytetrafluoroethylene (PTFE), polyetheretherketone (PEEK), polyethylene, ultra-high molecular polyethylene, Kraton (polyisoprene), PET (Polyethylene terephthalate) and acetal or from suitable metallic materials that include, but are not limited to, stainless steel, cobalt-chrome alloy, titanium, titanium alloy, or nickel-titanium shape memory alloys, among others.

The handles 76 and finger handle 78 can be fabricated from suitable metallic materials that include, but are not limited to, stainless steel, cobalt-chrome alloy, titanium, titanium alloy, or nickel-titanium shape memory alloys, among others. Suitable polymeric materials for handle 76 and finger handle 78 include, but are not limited to, polyetheretherketone (PEEK), ABS, Ultem, Kraton (polyisoprene), PET (Polyethylene terephthalate) and acetal. Suitable non-degradable materials for the handle 76, include, but are not limited to, polyetheretherketone (PEEK), ABS, Ultem, Kraton (polyisoprene), PET (Polyethylene terephthalate) and acetal.

The distal loop 74, when fully expanded within the removable control element disc space 64, can provide a circumference measurement in centimeters (cm) of the dissected disc space 64. The distal loop can also be rotated 90 degrees and then can provide a height measurement in centimeters (cm).

FIG. 9 is a perspective view of a disc space volume chart 80 for use with the removable control element 70. The disc space volume chart 80 has a heading for estimating the disc volume in cubic centimeters (cc) 82. The chart 80 uses the disk height (mm) 84 and disc circumference (cm) 85 to estimate the circumference measurement in centimeters (cm) of the disc space 64 using the formula for the volume of a cylinder v=πr² h where:

V=the volume of a cylinder
Π=Pi=3.14 constant
R=radius of the cylinder or disc space
H=height of the cylinder or disc space
Since, the circumference (c)=2πr; the volume of the cylinder of disc space is estimated by

$$V = \frac{{C}^{2} ( h )}{4 π}$$

It is anticipated by the Applicants' that this disc volume chart 80 can be included in a specific card or with instructions for use within the clinical kit or could be printed on one of the components in the clinical kit.

FIG. 10 is a partial cross-section view of the distal end the present invention showing the fixed-sized substantially flattened loop shaped mesh or toroidal shaped mesh 10 in a folded configuration 11 and inserted within the distal end of the delivery sheath 100 resulting in a delivery configuration. Also shown in the cross-section is the fill/delivery tubular member 36 with the locking jaws 32 and terminal raised tabs 42.

FIG. 11 is a partial cross-sectional view of the delivery configuration 11 of the present invention in position to be inserted into an access hole 66 within an intervertebral disc 60 having a plurality of spinal nerves. The delivery configuration of the delivery sheath 100 is shown with the contracted and folded sewn mesh 11 engaged to the fill/delivery tubular member 36 having a pair of locking jaws 32 and terminal raised tabs 42. Once the contracted and folded sewn mesh 11 is fully inserted within the intervertebral disc space 64, the delivery sheath 100 is retracted allowing the contracted sewn mesh loop 11 to expand within the disc space 64.

FIG. 12 is a partial cross-section view of the present invention with substantially flattened loop shaped or toroidal shaped mesh 10 deployed within the disc space 64 and the removable control element 70 inserted though the fill/delivery tubular member 36 inside the substantially flattened loop shaped mesh or toroidal shaped mesh 10 and deployed to further expand the substantially flattened loop shaped mesh or toroidal shaped mesh 10 against the inner wall of the dissected disc space 64. Advancing the handle 76 of the removable control element 70 expands the distal loop 74 circumferentially within the disc space 64 engaging the substantially flattened loop shaped mesh or toroidal shaped mesh 10 to the disc space wall. Applicants' anticipate that the flexible balloon similar to that shown in FIG. 7 could also be used to confirm expansion of or alternately expand the substantially flattened loop shaped mesh or toroidal shaped mesh 10 against the inner wall of the disc space 64.

FIG. 13 is a partial cross-section view of the present invention substantially flattened loop shaped mesh or toroidal shaped mesh 10 fully expand within the disc space 64 and showing a series of autograft or allograft fill cartridges 100a, 100b, and 100x filled with an autograft bone material or allograft 102a, 102b, and 102x. The fill tube cartridges 100a, 100b and 100x are used with the delivery tamp member 103 which is labeled with a length denoting the fill length of the autograft or allograft material within the cartridges 100a, 100b, 100x. By using the estimated volume of the disc space, one or more cartridges are used to deliver a specific volume of autograft bone material 102a, 102b or 100 to fill the central area of the expanded substantially flattened loop shaped mesh or toroidal shaped mesh 10. It is anticipated by the Applicants' that other biocompatible materials, such as one or more materials selected from the group consisting of hydrophilic polymers, hydrogels, homopolymer hydrogels, copolymer hydrogels, multi-polymer hydrogels, or interpenetrating hydrogels, acrylonitrile, acrylic acid, acrylimide, acrylimidine, including but not limited to PVA, PV, PHEMA, PNVP, polyacrylamides, poly (ethylene oxide), polyvinyl alcohol, polyarylonitrile, and polyvinyl pyrrolidone, silicone, polyurethanes, polycarbonate polyurethane (e.g, Corethane) other biocompatibile polymers, or combinations thereof, could be used to fill the central area of the expanded substantially flattened loop shaped mesh or toroidal shaped mesh 10. It is also anticipated that biocompatible materials formed of a material that is allowed to expand through the adsorption of liquids such as water selected from the group consisting of polyacrliamide, polyacrylonitrile, polyvinyl alcohol or other biocompatible hydrogels, solid fibrous collagen or other suitable hydrophilic biocompatible material or combinations thereof, could be used to fill the central area of the expanded substantially flattened loop shaped mesh or toroidal shaped mesh 10. In addition, it is anticipated by the Applicants' that biocompatible materials selected from the group consisting of steroids, antibiotics, tumor necrosis factor alpha or its antagonists, analgesics, growth factors, genes or gene vectors in solution; biologic materials (hyaluronic acid, noncrosslinked collagen, fibrin, liquid fat or oils); synthetic polymers (polyethylene glycol, liquid silicones, synthetic oils), saline or combinations thereof could be used to fill the central area of the expanded substantially flattened loop shaped mesh or toroidal shaped mesh 10. Furthermore, it is anticipated that the expanded substantially flattened loop shaped mesh or toroidal shaped mesh 10 could be filled with biocompatible material selected from the group consisting of bone graft materials such as any described “bone cements” or any polymeric bone graft compounds, bone graft materials, bone chips, nylon fibers, carbon fibers, glass fibers, collagen fibers, ceramic fibers, polyethylene fibers, polypropylene fibers, poly(ethylene terephthalate), polyglycolides, polylactides, and combinations thereof. It is further anticipated that biocompatible material is formed from calcium phosphate-based bone substitutes such as monolithic tetracalcium phosphate (CA₄(PO₄)₂0), Na₃PO₄; Na₂HPO₄; NaH₂PO₄; Na₄HPO₄.7H₂O; Na₃PO₄.12H₂O; H₃PO₄; CaSO₄; (NH₄)₃PO₄; (NH₄)₂HPO₄; (NH₄)H₂PO₄; (NH₄)₃PO₄.3H₂O; NaHCO₃; CaCO3; Na₂CO₃; KH₂PO₄; K₂HPO₄; K₃PO₄; CaF₂:SrF₂; Na₂SiF₆; Na₂PO₃F, and combinations thereof. Furthermore, it is anticipated that an amount of one or more active agents suitable to promote bone growth, such as a growth factor, BMP, a bone morphology protein, or a pharmaceutical carrier, and combination thereof could be use alone or in conjunction with another biocompatible material to fill the central area of the expanded substantially flattened loop shaped mesh or toroidal shaped mesh 10.

FIG. 14 is a partial cross-section of a treated, intervertebral disc 60 having a plurality of spinal nerves 62. The treated intervertebral disc 60 is shown with the substantially flattened loop shaped mesh or toroidal shaped mesh 10 central area filled with delivered autograft or autograft material 103 within the disc space 64. The disc access hole 66 is void of the fill/delivery tubular member which has been retracted after delivery completion of the specific volume of the autograft bone material or allograft 103.

FIG. 15 is a flowchart 110 depicting the general sequence of steps used with the present invention and its accessory components. Box 112 represents accessing the intervertebral disc by standard hospital procedures. After obtaining access to the intervertebral disc, Box 114 requires a standard discectomy procedure. After the discectomy procedure is complete, Box 116 represents measuring the disc circumference using the removable control element 70. Box 118 represents selected the correct substantially flattened loop shaped mesh or toroidal shaped mesh 10 for the measured disc circumference. Box 120 represents loading the contracted fixed mesh loop 11 into the delivery sheath. Box 122 represents inserting the contracted fixed sized mesh loop 11 into the intervertebral disc space through the access hole 66. Box 124 represents retracting and removing the delivery sheath 100 and then Box 126 represents the functionality of the present invention whereby the substantially flattened loop shaped mesh or toroidal shaped mesh 10 partially self expands within the disc space 64. Box 128 defines the method of fully expanding the substantially flattened loop shaped mesh or toroidal shaped mesh 10 using the removable control element Box 130 represents the optional procedures of inserting a flexible balloon 90 inside the intervertebral disc space 64 to confirm expansion of the substantially flattened loop shaped mesh or toroidal shaped mesh 10 within the disc space. The volume of the dissected disc space can also be estimated by the volume chart 80. Box 132 represents the procedure of loading the correct number of cartridges 102a, 102b, 102x with a autograph or allograft bone material. Box 134 represents the procedure of delivering the autograft or allograft material into the disc space cavity. Box 136 represents repeating the procedure defined in Box 134 until the disc space cavity is full. Box 138 defines the procedure of packing the autograft or allograft material as needed. Box 140 represents the method of releasing the collar jaws 32 and removing the delivery system from the substantially flattened loop shaped mesh or toroidal shaped mesh 10. Box 142 represents the procedure of manipulating the delivered and filled substantially flattened loop shaped mesh or toroidal shaped mesh 10 to close the delivery system opening. Box 144 represents the optional procedure of installing posterior stabilization 160 in the form of one or more pedicle screws 162 and/or one or more facet screws 150a, 150b. Box 146 represents the step of closing the treatment site using standard hospital procedures.

FIG. 16 is a partial sectional top view of the inter-vertebral disc 61 showing the expanded substantially flattened loop shaped mesh or toroidal shaped mesh 10 filled with autograft or allograft material 103 within the disc space 64 and two successive discs 61a, 61b affixed using a plurality of facet screws 150a, 150b. FIG. 17 is a partial sectional side view a first intervertebral disc 61a and a second intervertebral discs 61b showing the expanded substantially flattened loop shaped mesh or toroidal shaped mesh 10 filled with autograft or allograft material 103 and affixed with a plurality of facet screws 150a, 150b.

FIG. 18 is posterior view of an intervertebral disc showing the substantially flattened loop shaped mesh or toroidal shaped mesh 10 filled with autograft or allograft material 103 within the disc space 64 and two successive discs 61a, 61b affixed using a pair of pedicle screws 166 with stabilization bars 164 and pedicle screw nuts 166 for posterior support 160.

1. An apparatus for treating a spinal disc comprising:
a substantially flattened mesh apparatus, said mesh apparatus comprised by overlapping a portion of an outer mesh over a portion of an inner mesh creating an overlapped section, said overlapped section including a sewing means for engaging said outer mesh and inner mesh together in the overlapped section resulting in a fixed sized mesh, said fixed sized mesh having an interior chamber and an inner located hole section;
a collar tubular member;
a fill/delivery tubular member, said fill/delivery tubular member in coaxial association with said collar tubular member, said fixed size mesh removably engaged to said fill delivery tubular member.

2. The apparatus for treating a spinal disc as recited in claim 1, further including a removable/sizer control element.

3. The apparatus for treating a spinal disc as recited in claim 1, further including an expandable balloon designed to confirm the discectomy and expansion of the fixed sized mesh in the disc space.

4. The apparatus for treating a spinal disc as recited in claim 1, further including a expandable balloon designed to enhance the expansion of the fixed sized mesh within a spinal area.

5. The apparatus for treating a spinal disc as recited in claim 1, whereby one or more materials are delivered in said interior chamber of said mesh through said fill/delivery tubular member.

6. The apparatus for treating a spinal disc as recited in claim 4, whereby said one or more deliverable materials are delivered by air pressure.

7. The apparatus for treating a spinal disc as recited in claim 4, whereby said one or more deliverable materials are delivered by a mechanical means.

8. The apparatus for treating a spinal disc as recited in claim 4, whereby said one or more deliverable materials are delivered by a electro-mechanical means.

9. The apparatus for treating a spinal disc as recited in claim 4, whereby said one or more deliverable materials are delivered by a hydrodynamic means.

10. The apparatus for treating a spinal disc as recited in claim 1, further including a sheath for covering said fixed size mesh when in a contracted deliverable configuration.

11. The method of treating a diseased intervertebral disc as recited in claim 11, wherein said one or more materials are formed of a material selected from the group consisting of hydrophilic polymers, hydrogels, homopolymer hydrogels, copolymer hydrogels, multi-polymer hydrogels, or interpenetrating hydrogels, acrylonitrile, acrylic acid, acrylimide, acrylimidine, including but not limited to PVA, PVP, PHEMA, PNVP, polyacrylamides, poly(ethylene oxide), polyvinyl alcohol, polyarylonitrile, and polyvinyl pyrrolidone, silicone, polyurethanes, polycarbonate-polyurethane (e.g., Corethane) other biocompatibile polymers, or combinations thereof.

13. The method of treating a diseased intervertebral disc as recited in claim 11, wherein said material is formed of a material that is allowed to expand through the adsorption of liquids such as water selected from the group consisting of polyacrliamide, polyacrylonitrile, polyvinyl alcohol or other biocompatible hydrogels, solid fibrous collagen or other suitable hydrophilic biocompatible material or combinations thereof.

14. The method of treating a diseased intervertebral disc as recited in claim 11, wherein said material is formed of a material selected from the group consisting of steroids, antibiotics, tumor necrosis factor alpha or its antagonists, analgesics, growth factors, genes or gene vectors in solution; biologic materials (hyaluronic acid, non-crosslinked collagen, fibrin, liquid fat or oils); synthetic polymers (polyethylene glycol, liquid silicones, synthetic oils), saline or combinations thereof.

15. The method of treating a diseased intervertebral disc as recited in claim 11, wherein said material is formed of a material selected from the group consisting of bone graft materials such as any described “bone cements” or any polymeric bone graft compounds, bone graft materials, bone chips, nylon fibers, carbon fibers, glass fibers, collagen fibers, ceramic fibers, polyethylene fibers, polypropylene fibers, poly(ethylene terephthalate), polyglycolides, polylactides, and combinations thereof.

16. The method of treating a diseased intervertebral disc as recited in claim 11, wherein said material is formed from calcium phosphate-based bone substitutes such as monolithic tetracalcium phosphate (CA₄(PO₄)₂O).

17. The method of treating a diseased intervertebral disc as recited in claim 16, further comprising additional substances, such as Na₃PO₄; Na₂HPO₄; NaH₂PO₄; Na₄HPO₄. 7H₂O; Na₃PO₄.12H₂O; H₃PO₄; CaSO₄; (NH₄)₃PO₄; (NH₄)₂HPO₄; (NH₄)H₂PO₄; (NH₄)₃PO₄. 3H₂O; NaHCO₃; CaCO3; Na₂CO₃; KH₂PO₄; K₂HPO₄; K₃PO₄; CaF₂:SrF₂; Na₂SiF₆; Na₂PO₃F, and combinations thereof.

18. The method of treating a diseased intervertebral disc as recited in claim 11, further comprising an amount of one or more active agents suitable to promote bone growth, such as a growth factor, BMP, a bone morphology protein, or a pharmaceutical carrier, and combination thereof.

19. The method of treating a diseased intervertebral disc as recited in claim 16, further comprising an amount of one or more active agents suitable to promote bone growth, such as a growth factor, BMP, a bone morphology protein, or a pharmaceutical carrier, and combination thereof.

20. The method of treating a diseased intervertebral disc as recited in claim 11, after the step of retracting the introducer tubular member inserting a removable/sizer control element to enhance the expansion of the substantially flattened loop shaped mesh or toroidal shaped mesh within the disc space.

21. The method of treating a diseased intervertebral disc as recited in claim 11, after the step of retracting the introducer tubular member inserting a removable/sizer control element to provide volumetric measurements of a spinal area

22. The method of treating a diseased intervertebral disc as recited in claim 11, after the step of retracting the introducer tubular member inserting a expandable balloon designed to provide volumetric measurements of a spinal area.

23. The method of treating a diseased intervertebral disc as recited in claim 11, after the step of retracting the introducer tubular member further including a expandable balloon designed to enhance the expansion of the substantially flattened loop shaped mesh or toroidal shaped mesh within a spinal area.

24. A method of treating a diseased intervertebral disc, comprising the steps of:
creating an access opening in a disc between the adjacent vertebrae;
removing at least a portion of a nucleus within said disc which results in forming a cavity surrounded by a portion of the annulus of said disc;
advancing into a cavity an expandable substantially flattened loop shaped mesh or toroidal shaped mesh having an interior section and outer section, said expandable braided mesh attached to a collar member through an insertion hole created in said mesh, said collar member having an inner lumen;
advancing a tubular member with an inner member and distal expandable loop into the inner lumen of said collar member whereby said tubular member, inner member and distal expandable loop expand said substantially flattened loop shaped mesh or toroidal shaped mesh in the said cavity;
retracting said tubular member;
delivering one or more materials through said collar inner lumen in said substantially flattened loop shaped mesh or toroidal shaped mesh and cavity;
detaching said collar member whereby the insertion hole in the substantially flattened loop shaped mesh or toroidal shaped mesh is self-sealed.

25. The method of treating a diseased intervertebral disc as recited in claim 24, wherein said one or more materials are formed of a material selected from the group consisting of hydrophilic polymers, hydrogels, homopolymer hydrogels, copolymer hydrogels, multi-polymer hydrogels, or interpenetrating hydrogels, acrylonitrile, acrylic acid, acrylimide, acrylimidine, including but not limited to PVA, PVP, PHEMA, PNVP, polyacrylamides, poly(ethylene oxide), polyvinyl alcohol, polyarylonitrile, and polyvinyl pyrrolidone, silicone, polyurethanes, polycarbonate-polyurethane (e.g., Corethane) other biocompatibile polymers, or combinations thereof.

26. The method of treating a diseased intervertebral disc as recited in claim 24, wherein said material is formed of a material that is allowed to expand through the adsorption of liquids such as water selected from the group consisting of polyacrliamide, polyacrylonitrile, polyvinyl alcohol or other biocompatible hydrogels, solid fibrous collagen or other suitable hydrophilic biocompatible material or combinations thereof.

27. The method of treating a diseased intervertebral disc as recited in claim 24, wherein said material is formed of a material selected from the group consisting of steroids, antibiotics, tumor necrosis factor alpha or its antagonists, analgesics, growth factors, genes or gene vectors in solution; biologic materials (hyaluronic acid, non-crosslinked collagen, fibrin, liquid fat or oils); synthetic polymers (polyethylene glycol, liquid silicones, synthetic oils), saline or combinations thereof.

28. The method of treating a diseased intervertebral disc as recited in claim 24, wherein said material is formed of a material selected from the group consisting of bone graft materials such as any described “bone cements” or any polymeric bone graft compounds, bone graft materials, bone chips, nylon fibers, carbon fibers, glass fibers, collagen fibers, ceramic fibers, polyethylene fibers, polypropylene fibers, poly(ethylene terephthalate), polyglycolides, polylactides, and combinations thereof.

29. The method of treating a diseased intervertebral disc as recited in claim 24, wherein said material is formed from calcium phosphate-based bone substitutes such as monolithic tetracalcium phosphate (CA₄(PO₄)₂O).

30. The method of treating a diseased intervertebral disc as recited in claim 29, further comprising additional substances, such as Na₃PO₄; Na₂HPO₄; NaH₂PO₄; Na₄HPO₄. 7H₂O; Na₃PO₄.12H₂O; H₃PO₄; CaSO₄; (NH₄)₃PO₄; (NH₄)₂HPO₄; (NH₄) H₂PO₄; (NH₄)₃PO₄.3H₂O; NaHCO₃; CaCO3; Na₂CO₃; KH₂PO₄; K₂HPO₄; K₃PO₄; CaF₂: SrF₂; Na₂SiF₆; Na₂PO₃F, and combinations thereof.

31. The method of treating a diseased intervertebral disc as recited in claim 24, further comprising an amount of one or more active agents suitable to promote bone growth, such as a growth factor, BMP, a bone morphology protein, or a pharmaceutical carrier, and combination thereof.

32. The method of treating a diseased intervertebral disc as recited in claim 29, further comprising an amount of one or more active agents suitable to promote bone growth, such as a growth factor, BMP, a bone morphology protein, or a pharmaceutical carrier, and combination thereof.

33. The method of treating a diseased intervertebral disc as recited in claim 24, after the step of retracting the introducer tubular member inserting a removable/sizer control element to enhance the expansion of the substantially flattened loop shaped mesh or toroidal shaped mesh within the disc space.

34. The method of treating a diseased intervertebral disc as recited in claim 24, after the step of retracting the introducer tubular member inserting a removable/sizer control element to provide volumetric measurements of a spinal area

35. The method of treating a diseased intervertebral disc as recited in claim 24, after the step of retracting the introducer tubular member inserting a expandable balloon designed to provide volumetric measurements of a spinal area.

36. The method of treating a diseased intervertebral disc as recited in claim 24, after the step of retracting the introducer tubular member further including a expandable balloon designed to enhance the expansion of the substantially flattened loop shaped mesh or toroidal shaped mesh within a spinal area.
